(19) United States
(12) Patent Application Publication
(10) Pub. No.: US 2006/0046252 A1
(43) Pub. Date: Mar. 2, 2006

(54) Method and system for developing probes for dye normalization of microarray signal-intensity data

(76) Inventors: Srinka Ghosh, San Francisco, CA (US); Patrick J. Collins, San Francisco, CA (US)

Correspondence Address: AGILENT TECHNOLOGIES, INC.;Legal Department, DL429, Intellectual Property Administration, P. O. Box 7599, Loveland, CO, 80537-0599, US

(21) Appl. No.: 10/929,848

(22) Filed: Aug. 30, 2004

(51) Int. Cl. C12Q 1/68; C12P 19/34

(52) U.S. Cl. 435/6; 435/91.2

(57) ABSTRACT

A method and system for determining a set of dye-normalization microarray probes that consistently hybridize to approximately the same number of target molecules in a wide range of sample solutions. The method of one embodiment of the method of the present invention generates a set of candidate probe molecules. The set of candidate probe molecules are arrayed on one or more replicate microarrays. Sample solutions are made from one or more tissues of one or more species. Microarray-base hybridization assays are conducted by using the replicate microarrays and different sample solutions. A subset of the candidate probe molecules that are functional for the microarray-base hybridization assays are determined.

Embodiments of the present invention are related to microarray probes, and, in particular, to a method for determining a set of dye-normalization probes that consistently hybridize with target molecules over a wide range of species, tissues, and hybridization conditions.

### BACKGROUND OF THE INVENTION

The present invention is related to microarrays. In order to facilitate discussion of the present invention, a general background for particular kinds of microarrays is provided below. In the following discussion, the terms “microarray,” “molecular array,” and “array” are used interchangeably. The terms “microarray” and “molecular array” are well known and well understood in the scientific community. As discussed below, a microarray is a precisely manufactured tool which may be used in research, diagnostic testing, or various other analytical techniques to analyze complex solutions of any type of molecule that can be optically or radiometrically detected and that can bind with high specificity to complementary molecules synthesized within, or bound to, discrete features on the surface of a microarray. Because microarrays are widely used for analysis of nucleic acid samples, the following background information on microarrays is introduced in the context of analysis of nucleic acid solutions following a brief background of nucleic acid chemistry.

Deoxyribonucleic acid (“DNA”) and ribonucleic acid (“RNA”) are linear polymers, each synthesized from four different types of subunit molecules. FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108. Phosphorylated subunits of DNA and RNA molecules, called “nucleotides,” are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′ end to the 3′ end, the single letter abbreviations A, T, C, and G for the nucleotide subunits that together compose the DNA polymer. For example, the oligomer 100 shown in FIG. 1 can be chemically represented as “ATCG.”

The DNA polymers that contain the organization information for living organisms occur in the nuclei of cells in pairs, forming double-stranded DNA helices. One polymer of the pair is laid out in a 5′ to 3′ direction, and the other polymer of the pair is laid out in a 3′ to 5′ direction, or, in other words, the two strands are anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. FIGS. 2A-B illustrates the hydrogen bonding between the purine and pyrimidine bases of two anti-parallel DNA strands. AT and GC base pairs, illustrated in FIGS. 2A-B, are known as Watson-Crick (“WC”) base pairs. Two DNA strands linked together by hydrogen bonds forms the familiar helix structure of a double-stranded DNA helix. FIG. 3 illustrates a short section of a DNA double helix 300 comprising a first strand 302 and a second, anti-parallel strand 304.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary bases of anti-parallel DNA strands form WC base pairs in a cooperative fashion, leading to reannealing of the DNA duplex.

FIGS. 4-7 illustrate the principle of the microarray-based hybridization assay. A microarray (402 in FIG. 4) comprises a substrate upon which a regular pattern of features is prepared by various manufacturing processes. The microarray 402 in FIG. 4, and in subsequent FIGS. 5-7, has a grid-like 2-dimensional pattern of square features, such as feature 404 shown in the upper left-hand corner of the microarray. Each feature of the microarray contains a large number of identical oligonucleotides covalently bound to the surface of the feature. These bound oligonucleotides are known as probes. In general, chemically distinct probes are bound to the different features of a microarray, so that each feature corresponds to a particular nucleotide sequence.

Once a microarray has been prepared, the microarray may be exposed to a sample solution of target DNA or RNA molecules (410-413 in FIG. 4) labeled with fluorophores, chemiluminescent compounds, or radioactive atoms 415-418. Labeled target DNA or RNA hybridizes through base pairing interactions to the complementary probe DNA, synthesized on the surface of the microarray. FIG. 5 shows a number of such target molecules 502-504 hybridized to complementary probes 505-507, which are in turn bound to the surface of the microarray 402. Targets, such as labeled DNA molecules 508 and 509, that do not contain nucleotide sequences complementary to any of the probes bound to the microarray surface do not hybridize to generate stable duplexes and, as a result, tend to remain in solution. The sample solution is then rinsed from the surface of the microarray, washing away any unbound-labeled DNA molecules. In other embodiments, unlabeled target sample is allowed to hybridize with the microarray first. Typically, such a target sample has been modified with a chemical moiety that will react with a second chemical moiety in subsequent steps. Then, either before or after a wash step, a solution containing the second chemical moiety bound to a label is reacted with the target on the microarray. After washing, the microarray is ready for analysis. Biotin and avidin represent an example of a pair of chemical moieties that can be utilized for such steps.

Finally, as shown in FIG. 6, the bound labeled DNA molecules are detected via optical or radiometric instrumental detection. Optical detection involves exciting labels of bound labeled DNA molecules with electromagnetic radiation of appropriate frequency and detecting fluorescent emissions from the labels, or detecting light emitted from chemiluminescent labels. When radioisotope labels are employed, radiometric detection can be used to detect the signal emitted from the hybridized features. Additional types of signals are also possible, including electrical signals generated by electrical properties of bound target molecules, magnetic properties of bound target molecules, and other such physical properties of bound target molecules that can produce a detectable signal. Optical, radiometric, or other types of instrumental detection produce an analog or digital representation of the microarray as shown in FIG. 7, with features to which labeled target molecules are hybridized similar to 702 optically or digitally differentiated from those features to which no labeled DNA molecules are bound. Features displaying positive signals in the analog or digital representation indicate the presence of DNA molecules with complementary nucleotide sequences in the original sample solution. Moreover, the signal intensity produced by a feature is generally related to the amount of labeled DNA bound to the feature, in turn related to the concentration, in the sample to which the microarray was exposed, of labeled DNA complementary to the oligonucleotide within the feature.

One, two, or more than two data subsets within a data set can be obtained from a single microarray by scanning or reading the microarray for one, two or more than two types of signals. Two or more data subsets can also be obtained by combining data from two different arrays. When optical detection is used to detect fluorescent or chemiluminescent emission from chromophore labels, a first set of signals, or data subset, may be generated by reading the microarray at a first optical wavelength, a second set of signals, or data subset, may be generated by reading the microarray at a second optical wavelength, and additional sets of signals may be generated by detection or reading the microarray at additional optical wavelengths. Different signals may be obtained from a microarray by radiometric detection of radioactive emissions at one, two, or more than two different energy levels. Target molecules may be labeled with either a first chromophore that emits light at a first wavelength, or a second chromophore that emits light at a second wavelength. Following hybridization, the microarray can be read at the first wavelength to detect target molecules, labeled with the first chromophore, hybridized to features of the microarray, and can then be read at the second wavelength to detect target molecules, labeled with the second chromophore, hybridized to the features of the microarray. In one common microarray system, the first chromophore emits light at a near infrared wavelength, and the second chromophore emits light at a yellow visible-light wavelength, although these two chromophores, and corresponding signals, are referred to as “red” and “green.” The data set obtained from reading the microarray at the red wavelength is referred to as the “red signal,” and the data set obtained from reading the microarray at the green wavelength is referred to as the “green signal.” While it is common to use one or two different chromophores, it is possible to use one, three, four, or more than four different chromophores and to read a microarray at one, three, four, or more than four wavelengths to produce one, three, four, or more than four data sets. With the use of quantum-dot dye particles, the emission is tunable by suitable engineering of the quantum-dot dye particles, and a fairly large set of such quantum-dot dye particles can be excited with a single-color, single-laser-based excitation.

Microarray data processing may reveal systematic variation in the different data sets produced for a single microarray or across several microarrays. As one example, intensities obtained from a green-labeled sample may be of larger magnitude, in general, than intensities obtained from a red-labeled sample of the red and green chromophores. The differences in signal intensities may be produced by differing labeling efficiencies, differences in the power of electromagnetic radiation used to excite the different labels, differing amounts of target molecules labeled in the different channels, or spatial biases in ratios across the surface of the microarray. Researchers, microarray designers, and manufacturers of microarrays and microarray data processing systems have therefore recognized a need for a reliable and efficient method for determining a set of dye normalizing probes that can be used to normalize intensity data generated from analysis of microarrays.

### SUMMARY OF THE INVENTION

Various embodiments of the present invention are directed to methods for determining a set of dye-normalization probes that consistently hybridize to approximately identical numbers of target molecules in a wide range of sample solutions. One embodiment of the method of the present invention generates a set of candidate probe molecules. The set of candidate probe molecules are arrayed on one or more replicate microarrays. Sample solutions are made from one or more tissues of one or more species. Microarray-base hybridization assays are conducted by using the replicate microarrays and different sample solutions. A subset of the candidate probe molecules that are functional for the microarray-base hybridization assays are determined.

### BRIEF DESCRIPTION OF THE DRAWINGS

FIG. 1 illustrates a short DNA polymer.

FIGS. 2A-B illustrate the hydrogen bonding between the purine and pyrimidine bases of two anti-parallel DNA strands.

FIG. 3 illustrates a short section of a DNA double helix comprising a first strand and a second, anti-parallel strand.

FIG. 4 illustrates a grid-like, two-dimensional pattern of square features.

FIG. 5 shows a number of target molecules hybridized to complementary probes, which are in turn bound to the surface of the microarray.

FIG. 6 illustrates the bound labeled DNA molecules detected via optical or radiometric scanning.

FIG. 7 illustrates optical, radiometric, or other types of scanning produced by an analog or digital representation of the microarray.

FIGS. 8A-B illustrate red-signal intensity to green signal intensity ratio plots.

FIG. 9 illustrates a hypothetical microarray probe.

FIG. 10 illustrates examples of low-complexity, synthetic microarray probes.

FIG. 11 illustrates a hypothetical microarray having sixteen groups each having four replicate features.

FIG. 12 shows four replicate hypothetical microarrays.

FIG. 13 shows a number of hypothetical sample solutions that can be prepared for the 10 species listed in Table 1.

FIG. 14 illustrates five hypothetical sample solutions.

FIG. 15 illustrates four replicate microarrays exposed to a hypothetical sample solution.

FIG. 16 illustrates a hypothetical replicate microarray after exposure to a sample solution.

FIG. 17 illustrates a log-ratio data plot of a hypothetical target-molecule pair.

FIG. 18 illustrates a red-signal intensity to green-signal intensity plot for a hypothetical set of candidate probes that satisfy a tolerance interval, as shown in FIG. 17.

FIG. 19 is an illustration of an example 8-pack of microarrays.

FIG. 20 illustrates three kinds of probes employed in designing 8-pack microarrays.

FIG. 21 illustrates five, hypothetical sample solutions.

FIGS. 22A-B show two of many possible dye-normalization probe feature arrangements.

FIG. 23 is a control-flow diagram that represents one of many possible methods according to the present invention for determining a set of synthetic dye-normalization probes.

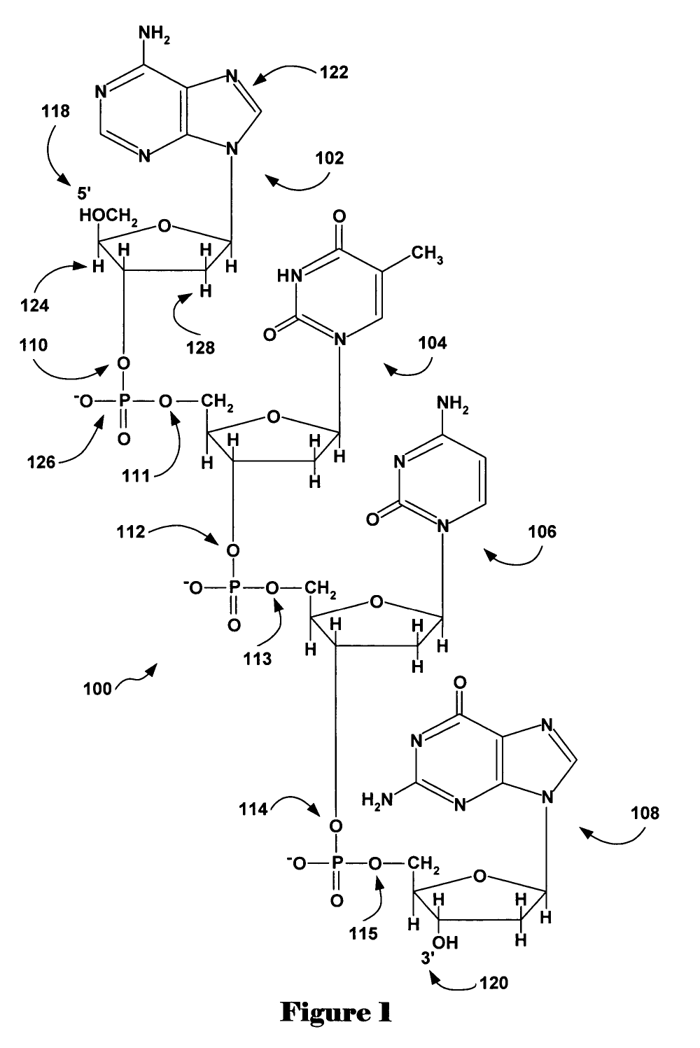

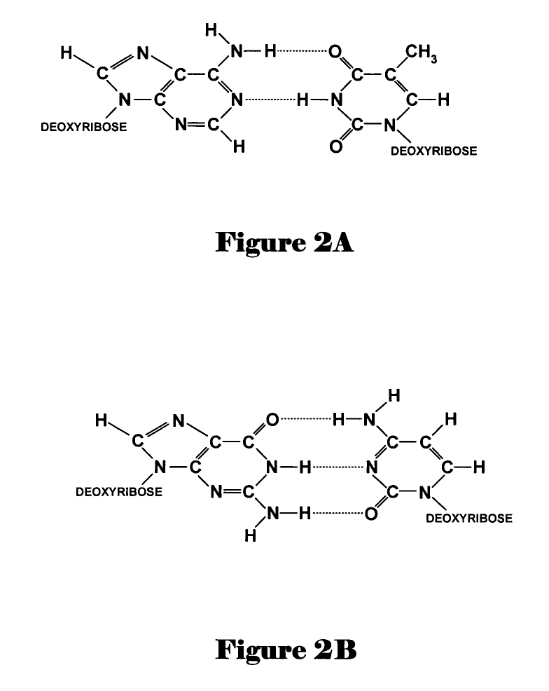

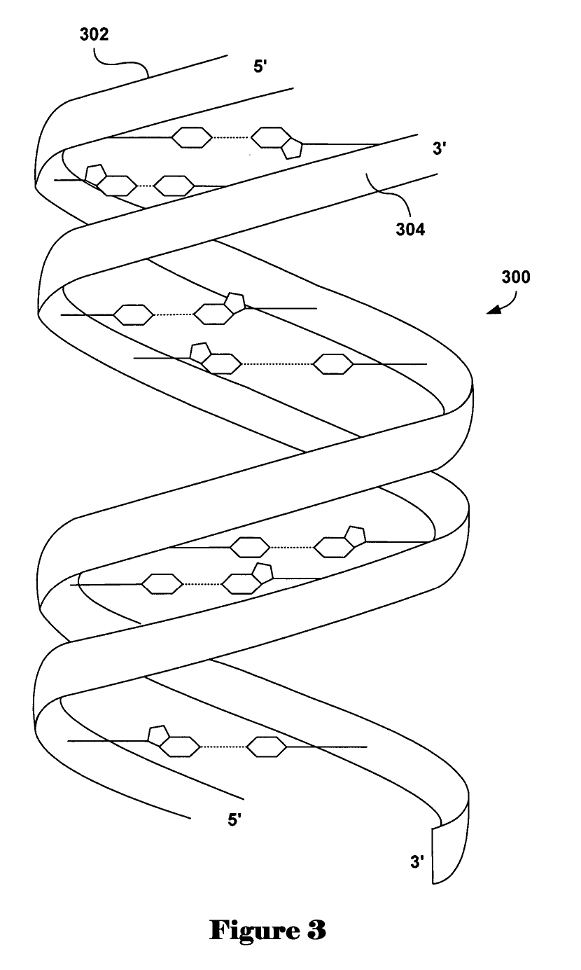

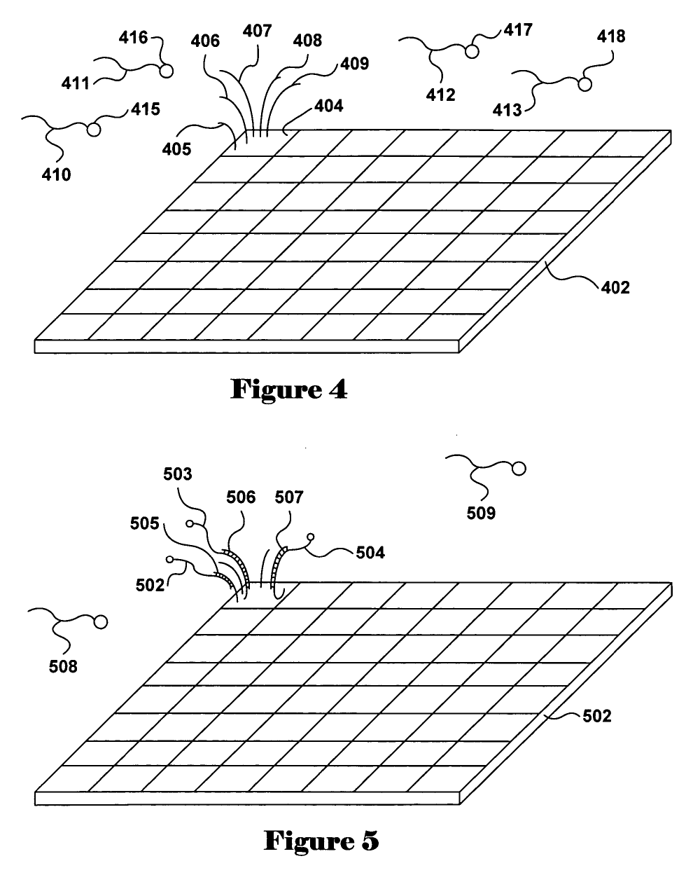

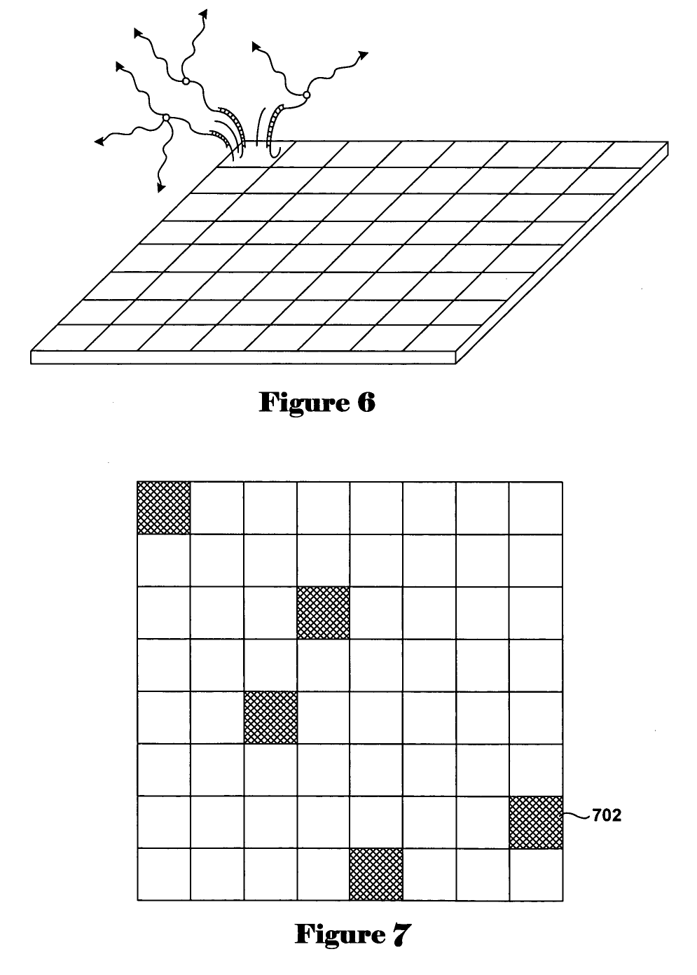

### DETAILED DESCRIPTION OF EMBODIMENTS OF THE INVENTION

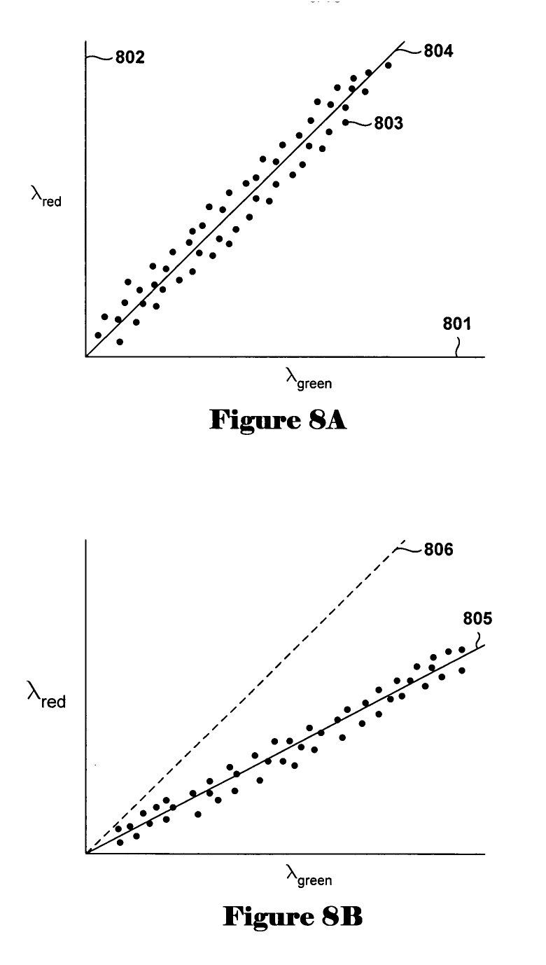

The present invention is directed to various types of synthetic microarray probes that span the entire intensity distribution of any given microarray experiment, consistently producing an intensity log ratio converging to “0” for different labels that hybridize with target molecules of a variety of species and tissues under various hybridization conditions. The following discussion includes two subsections, a first subsection including additional information about molecular arrays, a second subsection including additional information about dye-normalization probes, and a third subsection describing embodiments of the present invention with reference to FIGS. 8-23.

### Additional Information about Microarrays

A microarray may include any one-, two- or three-dimensional arrangement of addressable regions, or features, each bearing a particular chemical moiety or moieties, such as biopolymers, associated with that region. Any given microarray substrate may carry one, two, or four or more microarrays disposed on a front surface of the substrate. Depending upon the use, any or all of the microarrays may be the same or different from one another and each may contain multiple spots or features. A typical microarray may contain more than ten, more than one hundred, more than one thousand, more ten thousand features, or even more than one hundred thousand features, in an area of less than 20 cm² or even less than 10 cm². For example, square features may have widths, or round feature may have diameters, in the range from a 10 μm to 1.0 cm. In other embodiments each feature may have a width or diameter in the range of 1.0 μm to 1.0 mm, usually 5.0 μm to 500 μm, and more usually 10 μm to 200 μm. Features other than round or square may have area ranges equivalent to that of circular features with the foregoing diameter ranges. At least some, or all, of the features may be of different compositions (for example, when any repeats of each feature composition are excluded the remaining features may account for at least 5%, 10%, or 20% of the total number of features). Inter-feature areas are typically, but not necessarily, present. Inter-feature areas generally do not carry probe molecules. Such inter-feature areas typically are present where the microarrays are formed by processes involving drop deposition of reagents, but may not be present when, for example, photolithographic microarray fabrication processes are used. When present, interfeature areas can be of various sizes and configurations.

Each microarray may cover an area of less than 100 cm², or even less than 50 cm², 10 cm² or 1 cm². In many embodiments, the substrate carrying the one or more microarrays will be shaped generally as a rectangular solid having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. Other shapes are possible, as well. With microarrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Microarrays can be fabricated using drop deposition from pulsejets of either polynucleotide precursor units (such as monomers) in the case of in situ fabrication, or the previously obtained polynucleotide. Such methods are described in detail in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, photolithographic microarray fabrication methods may be used. Interfeature areas need not be present particularly when the microarrays are made by photolithographic methods as described in those patents.

A microarray is typically exposed to a sample including labeled target molecules, or, as mentioned above, to a sample including unlabeled target molecules followed by exposure to labeled molecules that bind to unlabeled target molecules bound to the microarray, and the microarray is then read. Reading of the microarray may be accomplished by illuminating the microarray and reading the location and intensity of resulting fluorescence at multiple regions on each feature of the microarray. For example, a scanner may be used for this purpose, which is similar to the AGILENT MICROARRAY SCANNER manufactured by Agilent Technologies, Palo Alto, Calif. Other suitable apparatus and methods are described in published U.S. patent applications 20030160183A1, 20020160369A1, 20040023224A1, and 20040021055A, as well as U.S. Pat. No. 6,406,849. However, microarrays may be read by any other method or apparatus than the foregoing, with other reading methods including other optical techniques, such as detecting chemiluminescent or electroluminescent labels, or electrical techniques, for where each feature is provided with an electrode to detect hybridization at that feature in a manner disclosed in U.S. Pat. No. 6,251,685, and elsewhere.

A result obtained from reading a microarray, followed by application of a method of the present invention, may be used in that form or may be further processed to generate a result such as that obtained by forming conclusions based on the pattern read from the microarray, such as whether or not a particular target sequence may have been present in the sample, or whether or not a pattern indicates a particular condition of an organism from which the sample came. A result of the reading, whether further processed or not, may be forwarded, such as by communication, to a remote location if desired, and received there for further use, such as for further processing. When one item is indicated as being remote from another, this is referenced that the two items are at least in different buildings, and may be at least one mile, ten miles, or at least one hundred miles apart. Communicating information references transmitting the data representing that information as electrical signals over a suitable communication channel, for example, over a private or public network. Forwarding an item refers to any means of getting the item from one location to the next, whether by physically tran-sporting that item or, in the case of data, physically transporting a medium carrying the data or communicating the data.

As pointed out above, microarray-based assays can involve other types of biopolymers, synthetic polymers, and other types of chemical entities. A biopolymer is a polymer of one or more types of repeating units. Biopolymers are typically found in biological systems and particularly include polysaccharides, peptides, and polynucleotides, as well as their analogs such as those compounds composed of, or containing, amino acid analogs or non-amino-acid groups, or nucleotide analogs or non-nucleotide groups. This includes polynucleotides in which the conventional backbone has been replaced with a non-naturally occurring or synthetic backbone, and nucleic acids, or synthetic or naturally occurring nucleic-acid analogs, in which one or more of the conventional bases has been replaced with a natural or synthetic group capable of participating in Watson-Crick-type hydrogen bonding interactions. Polynucleotides include single or multiple-stranded configurations, where one or more of the strands may or may not be completely aligned with another. For example, a biopolymer includes DNA, RNA, oligonucleotides, and PNA and other polynucleotides as described in U.S. Pat. No. 5,948,902 and references cited therein, regardless of the source. An oligonucleotide is a nucleotide multimer of about 10 to 100 nucleotides in length, while a polynucleotide includes a nucleotide multimer having any number of nucleotides.

As an example of a non-nucleic-acid-based microarray, protein antibodies may be attached to features of the microarray that would bind to soluble labeled antigens in a sample solution. Many other types of chemical assays may be facilitated by microarray technologies. For example, polysaccharides, glycoproteins, synthetic copolymers, including block copolymers, biopolymer-like polymers with synthetic or derivitized monomers or monomer linkages, and many other types of chemical or biochemical entities may serve as probe and target molecules for microarray-based analysis. A fundamental principle upon which microarrays are based is that of specific recognition, by probe molecules affixed to the microarray, of target molecules, whether by sequence-mediated binding affinities, binding affinities based on conformational or topological properties of probe and target molecules, or binding affinities based on spatial distribution of electrical charge on the surfaces of target and probe molecules.

Scanning of a microarray by an optical scanning device or radiometric scanning device generally produces an image comprising a rectilinear grid of pixels, with each pixel having a corresponding signal intensity. These signal intensities are processed by a microarray-data-processing program that analyzes data scanned from an microarray to produce experimental or diagnostic results which are stored in a computer-readable medium, transferred to an intercommunicating entity via electronic signals, printed in a human-readable format, or otherwise made available for further use. Microarray experiments can indicate precise gene-expression responses of organisms to drugs, other chemical and biological substances, environmental factors, and other effects. Microarray experiments can also be used to diagnose disease, for gene sequencing, and for analytical chemistry. Processing of microarray data can produce detailed chemical and biological analyses, disease diagnoses, and other information that can be stored in a computer-readable medium, transferred to an intercommunicating entity via electronic signals, printed in a human-readable format, or otherwise made available for further use.

### Additional Information about Dye-Normalization Probes

Multiple data sets may be obtained from a single microarray, and multiple microarrays can generate multiple data sets. These data sets have different meanings, depending on the different types of experiments in which the microarrays are exposed to target-molecule-containing solutions. Frequently, data sets read from multiple microarrays are experimentally related, and data sets read at different optical frequencies from a single microarray are commonly related to one another. However, in order to meaningfully analyze and compare multiple data sets, the multiple data sets need to be normalized with respect to one another.

FIGS. 8A-B illustrate red-channel-to-green-channel-ratio plots for two hypothetical microarray data sets. The hypothetical microarray data sets are obtained from two hypothetical microarray-based assays conducted with sample solutions composed of a variety of different target-molecule pairs, each pair composed of red-labeled and green-labeled target molecules having identical nucleotide sequences and concentrations. In the plots, the horizontal axes, such as horizontal axis 801, correspond to the green-signal intensities, and the vertical axes, such as vertical axis 802, correspond to the red-signal intensities. In FIGS. 8A-B, each data point, such as data point 803, corresponds to the ratio of red-signal intensity to green-signal intensity for a particular feature of a hypothetical microarray data set. The central tendency of the hypothetical data points plotted in FIG. 8A exhibit an ideal ratio of red-signal intensity to green-signal intensity for each feature, as indicated by line 804 having slope 1. A central tendency with slope “1” indicates a lack of apparent systematic intensity variation between channels. By contrast, data points in FIG. 8B correspond to a hypothetical microarray data set that exhibits differences in red labeling and green labeling efficiency, and is referred to as “dye-label bias.” In FIG. 8B, the central tendency of the data points, represented by line 805 having slope ½, indicate that the measured green signal is, for one of the various reasons discussed above, generally twice as intense as the measured red signal when an equal number of green and red chromophores are present. Dashed line 806 represents the ideal ratio of red-signal intensities to green-signal intensities having a slope equal to 1.

In general, dye-normalization probes are utilized in an attempt to normalize signal intensities, such as the systematic variation shown in FIG. 8B. Ideally, effective dye-normalization probes hybridize to target molecules in a variety of tissues or sample solutions with approximately equal efficiency and span the entire signal intensity range of any microarray experiment. However, dye-normalization probes typically fail to span the full range of intensity distribution for any given set of microarray experiments and may not be useful for normalizing many different microarray-based hybridization assays for a variety of species and tissues. This can be particularly problematic for low-feature-count microarrays, such as microarrays having fewer than 2,000 features.

### Embodiments of the Present Invention

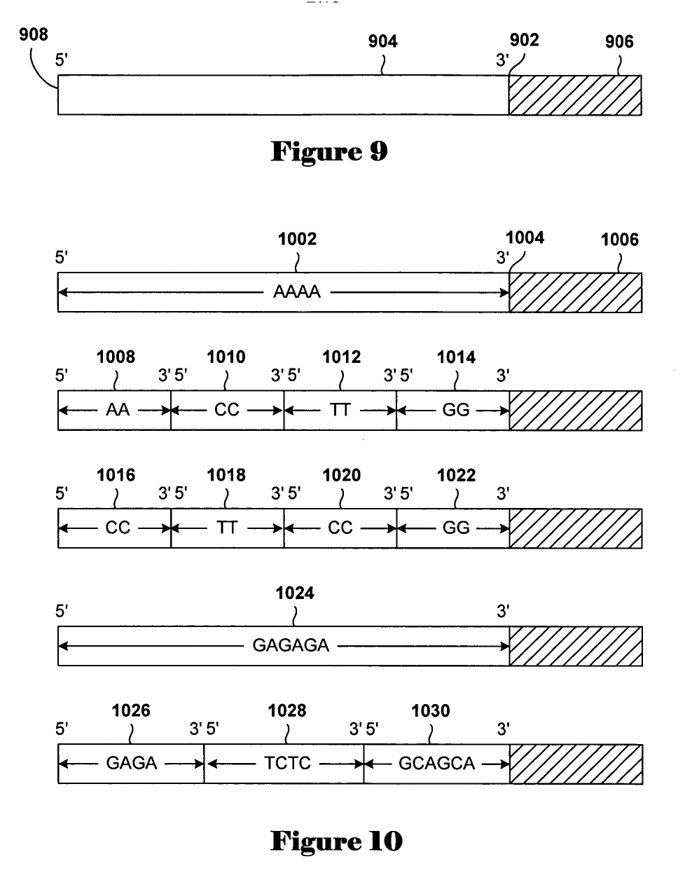

One of many possible embodiments of the present invention is directed to a method for determining a set of dye-normalization probes that consistently hybridize to approximately the same number of target molecules in a wide range of sample solutions and provide signal intensities that span most or all intensities of the entire intensity range of any microarray data set. An initial step of the method of the present invention is to generate a set of candidate probe molecules. A typical microarray probe can be notationally represented as:
[NS]ₙ-X-surface Equation (1):
where [NS]ₙ=a nucleotide sequence;
   n=number of nucleotides in the nucleotide sequence;
   surface=microarray surface; and
   X=an optional linker sequence of varying length that spaces the nucleotide sequence [NS]ₙ away from the surface.
      FIG. 9 is an illustration of a typical microarray probe having a linker sequence X that spaces the nucleotide sequence [NS]ₙ away from the microarray surface. In FIG. 9, the 3′-end 902 of the nucleotide sequence [NS]ₙ 904 is bound to the linker sequence X 906. The nucleotide sequence [NS]ₙ 904 is the portion of the probe responsible for hybridization with the complementary nucleotide sequence of a target molecule. Note that the nucleotide sequences [NS]ₙ may be oppositely oriented, with the 5′-end 908 bound to the linker sequence X rather than the 3′-end 902 bound to the linker sequence X.

In general, target molecules have complex nucleotide sequences. In other words, the target-molecule nucleotide sequence generally lacks discernable sequence patterns, and has relatively high information content, or high entropy. If the nucleotide sequence of a specific target molecule has already been determined, a probe can be designed for hybridization with a specific target molecule by synthesizing a complementary, complex nucleotide sequence [NS]ₙ. A probe designed to hybridize with a specific target molecule is unlikely to hybridize with other target molecules present in the sample solution, due to the low probability of a high entropy sequence of length about 8 or more occurring in two different target molecules. By contrast, the probe-design method of the present invention determines a set of candidate probe molecules that are likely to hybridize non-specifically with a wide variety of target molecules. The set of candidate probe molecules obtained by the embodiments of the present invention contains probes having low-complexity, low-entropy nucleotide sequences [NS]ₙ.

FIG. 10 illustrates examples of low-complexity, low-entropy candidate probe molecules. Nucleotide sequences [NS]ₙ that have one repeated nucleotide, such as [C]ₙ, [T]ₙ, [G]ₙ, and [A]ₙ, are the lowest-complexity nucleotides sequences, and are referred to as “homopolymers.” In FIG. 10, the nucleotide sequence 1002 is an example of a homopolymer [A]ₙ bound to the 3′-end 1004 of a linker sequence X 1006.

Shorter sequences of homopolymers can be bound together to generate additional kinds of low-complexity nucleotides sequences [NS]ₙ. For example, homopolymer sequences can be combined to give the following nucleotide sequence:
[NS]ₙ=[A]ᵢ[C]ⱼ[T]ₖ[G]ₗ Equation (2):
where i,j,k,l ε{1,2, . . . , n−3}; and
i+j+k+l=n
   The example nucleotide sequence [NS]ₙ given by equation (2) has four homopolymer subsequences [A]ᵢ 1008, [C]ⱼ 1010, [T]ₖ 1012, and [G]ₗ 1014.

The nucleotide sequence [NS]ₙ may be composed of repeating homopolymer subsequences:
[NS]ₙ=[C]ᵢ[T]ⱼ[C]ₖ[G]ₗ Equation (3):
where i+j+k+l=n
   The example nucleotide sequence [NS]ₙ given by equation (3) has four homoploymer subsequences [C]ᵢ 1016, [T]ⱼ 1018, [C]ₖ 1020, and [G]₁ 1022, where repeating cytosine homopolymer subsequences 1016 and 1020 are of different lengths i and j, respectively.

The low-complexity nucleotide sequences [NS]ₙ may also be composed of repeated subsequences, such as the following:
[GA]ₙ, [GAC]ₙ, [GACT]ₙ, . . . , [TC]ₙ, [TCA]ₙ, [TCAG]ₙ, . . .
   For example, in FIG. 10, the nucleic acid sequence [NS]ₙ 1024 is composed of a single repeating subsequence “GA.” Different repeating subsequences can also be combined to give nucleotide sequences of the form:
   [NS]ₙ=[GA]ᵢ[TC]ⱼ[GCA]ₖ Equation (4):
where i+j+k=n
   In FIG. 10, the example nucleic acid sequence [NS]ₙ given by equation (4) is composed of the repeated sequences [GA]ᵢ 1026, [TC]ⱼ 1028, and [GCA]ₖ 1030. The nucleotide sequence [NS]ₙ may also be composed of random nucleotide sequences combined with homopolymers and repeated subsequences.

In addition to varying the nucleotide sequence, as described above with reference to FIG. 10, the length n of the nucleotide sequences [NS]ₙ can be varied. The length n of the nucleotide sequence [NS]ₙ can range from about 25 to about 60 or more nucleotides. For example, nucleotide sequences of different lengths such as [GA]₃₀ and [A]₂₀ [TC]₁₂ [CAG]₂₄, can all be employed in a set of candidate probe molecules.

The microarray feature signal intensity can be modulated by varying the GC content of the nucleotide sequence [NS]ₙ. The higher the GC content, the more tightly the nucleotide sequences [NS]ₙ, will hybridize to non-specific target molecules in the sample solution. The set of candidate probe molecules can be expanded to include other low-complexity probes, such as low-complexity probes selected from Agilent's Human 1A Probe Selection Probe Database and probes synthesized from rat and mouse tissues, using the methods described in pending Agilent U.S. patent application Ser. No. 10/303,160 entitled “Methods for Identifying Suitable Nucleic Acid Normalization Probe Sequences for Use in Nucleic Acid Arrays,” filed Oct. 14, 2003, and Agilent U.S. patent application Ser. No. 10/686,092, entitled “Methods for Identifying Suitable Nucleic Acid Probe Sequences for Use in Nucleic Acid Arrays,” filed Nov. 22, 2003, which are incorporated by reference.

Subsequent steps of one method of the present invention identify “functional” candidate probe molecules. Functional candidate probe molecules consistently span the signal intensity range of a microarray, have a log ratio of approximately “0,” and hybridize with target molecules synthesized from different tissues of various species under a variety of hybridization conditions. Functional candidate probe molecules are determined by arraying a large number of candidate probe molecules on microarrays and conducting microarray-based hybridization assays with sample solutions having two or more different target molecules.

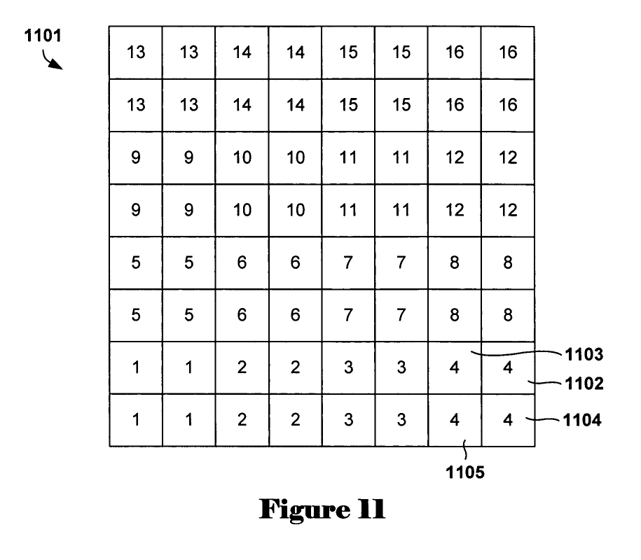

In an initial step, a microarray feature arrangement having from about 10,000 to about 22,000 or more different candidate probe molecules is designed. Typically, the microarray features are separated into different groups of one or more features, each group of one or more features having identical, candidate probe molecules. The one or more features having identical, candidate probe molecules are referred to as “replicate features.” FIG. 11 illustrates a hypothetical microarray 1101 having sixteen groups, each group having four replicate features. In FIG. 11, the groups of replicate features are numbered and occupy four adjacent features on the microarray surface. For example, the group of 4-replicate features 1102-1105 are identified by the number “4” and occupy the front-four, right-hand corner features of microarray 1101. Note that the present invention is not limited to the microarray-feature arrangement shown in FIG. 11. In other embodiments, the groups of replicate features can be arranged in a line, parallel with, or angled with respect to, an edge of the microarray, or scattered randomly over the surface of the microarray. Note further that, in other embodiments, the number of replicate features in a group of identical, candidate probe molecules may range from about 2 to about 20 or more microarray features.

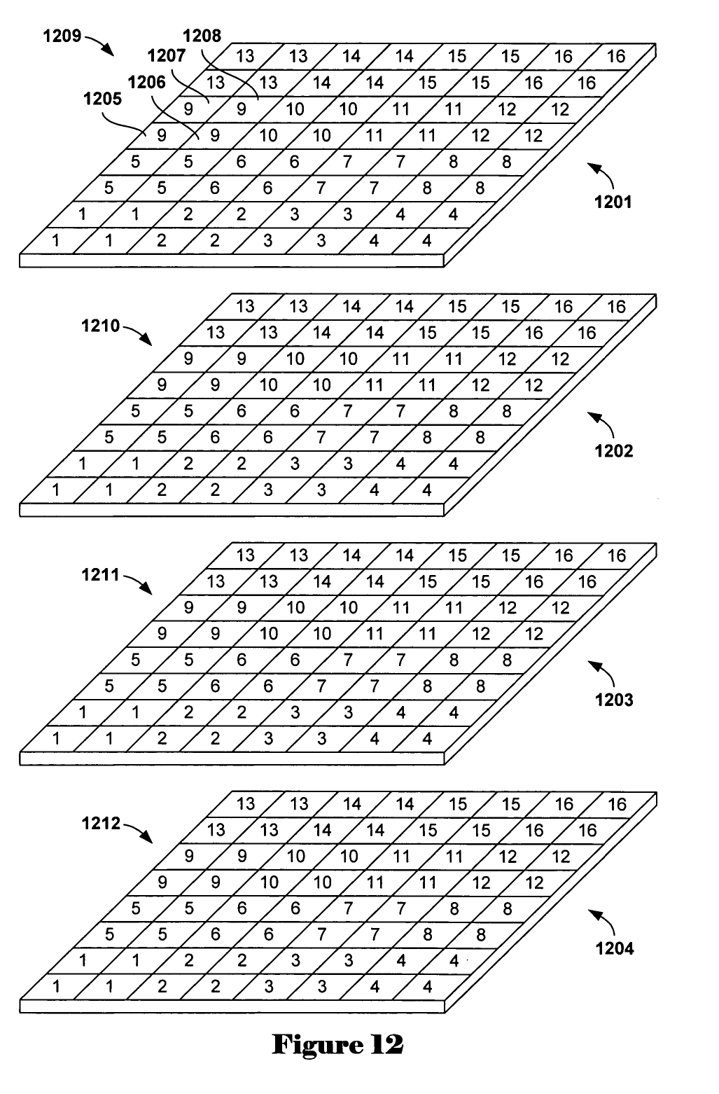

Next, a number of microarray-based hybridization assays are conducted using sets of two or more identical microarrays, each of which have identical arrangements of replicate features. The two or more identical microarrays are referred to as “replicate microarrays.” FIG. 12 shows four replicate, hypothetical microarrays. In FIG. 12, microarrays 1201-1204 each have identical feature arrangements. For example, replicate features 1205-1208, identified by the number “9,” are identical and occupy identical feature locations 1209-1212 on all four replicate microarrays 1201-1204.

The sample solutions used in the microarray-based hybridization assays are prepared by first selecting two or more species, and then selecting two or more tissues from each species. Table 1 displays a hypothetical set of ten possible species and a number of tissues used to determine the functionality of candidate probe molecules:

TABLE 1

| Species | Number of Tissues |
|---|---|
| Human | 10 |
| Rat | 10 |
| Mouse | 10 |
| Arabidopsis | 2 |
| Yeast | 2 |
| Rice | 2 |
| Wheat | 2 |
| Magnaporthe | 2 |
| Drosophila | 2 |
| C. elegans | 2 |

In Table 1, 10 different tissues are selected for the species “Human,” “Mouse,” and “Rat,” and 2 different tissues are selected for the remaining species listed. For example, the two different tissues selected for the species “Rice” may be the bran and grain tissues. Note that the present invention is not limited to the particular species nor to the number of species displayed in Table 1. In alternate embodiments, the number of different species may range from about 2 to about 20 or more, and the number of tissues selected for each species may range from about 2 to about 20 or more.

Next, target molecules for each sample solution are isolated from the nucleic acid molecules of each tissue. The target molecules can be either cDNA or amplified RNA copies of all expressed mRNA molecules in a given tissue. The target molecules synthesized from different tissues of a species are grouped in pairs called “target-molecule pairs.” Table 2 displays one of many possible target-molecule-pair combinations for the species “Human,” listed above in Table 1:

TABLE 2

| Target- | | |
|---|---|---|
| molecule | Target- | Dye |
| pair No. | molecule pair | Color |
| 1 | Lung | Red |
| | Heart | Green |
| 2 | Brain | Red |
| | Spleen | Green |
| 3 | Nerve | Red |
| | Muscle | Green |
| 4 | Liver | Red |
| | Intestine | Green |
| 5 | Pancreas | Red |
| | Placenta | Green |

In Table 2, target-molecule pair 1 is composed of target molecules isolated from lung and heart tissues. Note that the present invention is not limited to any particular set of tissues for determining target molecule nucleotide sequences. In alternate embodiments, an entirely different set of tissues can be selected. Note further that the present invention is not limited to the particular target-molecule pairs displayed in Table 2. For a species with 10 different tissues, such as the Human species, there are 45 possible target-molecule pair combinations. For example, target molecules extracted from lung tissue can be paired with target molecules extracted from liver tissue. The third column of Table 2 identifies the labels assigned to all target molecules of a particular tissue.

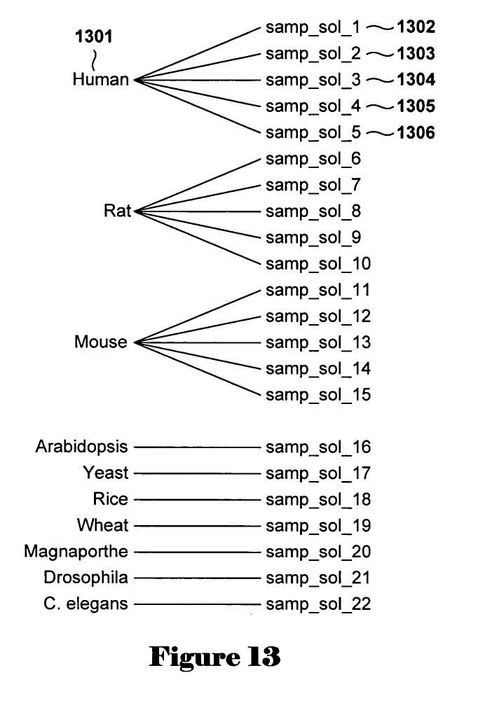

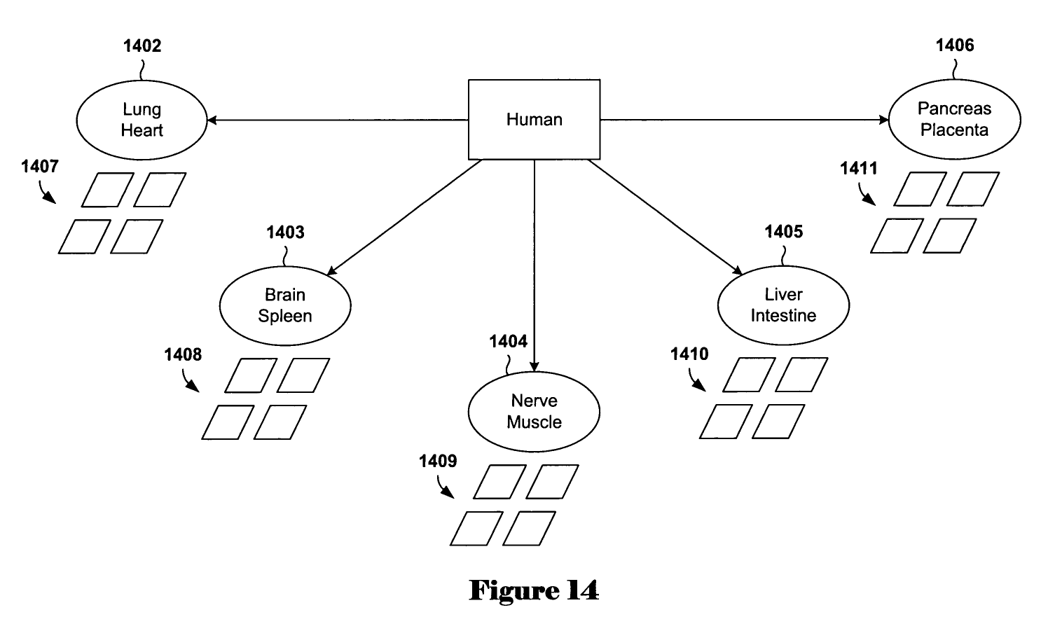

Next, for each target-molecule pair of each species, a separate sample solution is prepared. FIG. 13 shows hypothetical sample solutions that can be prepared for the 10 species listed in Table 1. In FIG. 13, the hypothetical sample solutions are labeled “samp_sol_1”-“samp_sol_22.” For example, the five, hypothetical, separate sample solutions 1302-1306 represent 5 of the 45 possible combinations of target-molecule pair sample solutions that can be prepared for the 10 tissues of the Human species 1301 displayed in Table 2. FIG. 14 illustrates five hypothetical sample solutions for the Human species 1401. In FIG. 14, hypothetical sample solutions 1402-1406 correspond to the target-molecule pairs listed in Table 2. For example, hypothetical sample solution 1402 is composed of lung and heart target molecules. For each sample solution, such as hypothetical sample solutions 1402-1406, a set of one or more replicate microarrays are prepared, as described above with reference to FIGS. 11 and 12. For example, five separate hypothetical microarray-based hybridization assays shown in FIG. 14 are performed by exposing each set of four replicate microarrays 1407-1411 to one of the sample solutions 1402-1406, respectively.

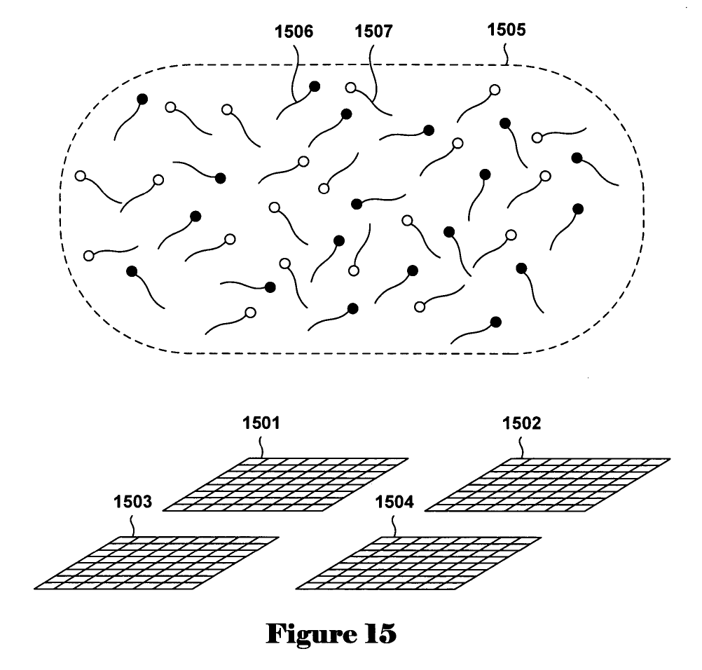

FIG. 15 illustrates a set of four replicate microarrays 1501-1504 exposed to the sample solution 1505, as described above with reference to FIG. 14. In FIG. 15, sample solution 1505 represents sample solution 1402 of FIG. 14 and target-molecule pair no. 1 of Table 2. Note that, for each sample solution, all target molecules that have been isolated from a first tissue of a species are labeled with an identical first signal emitting label, and all target molecules isolated from a second tissue of the same species are labeled with an identical second label that emits a signal different from that emitted by the first label. For example, in sample solution 1505, lung target molecules are labeled with red signal emitting labels, identified by shaded labels, such as target molecule 1506, and heart target molecules are labeled with green signal emitting labels, identified by unshaded labels, such as target molecule 1507.

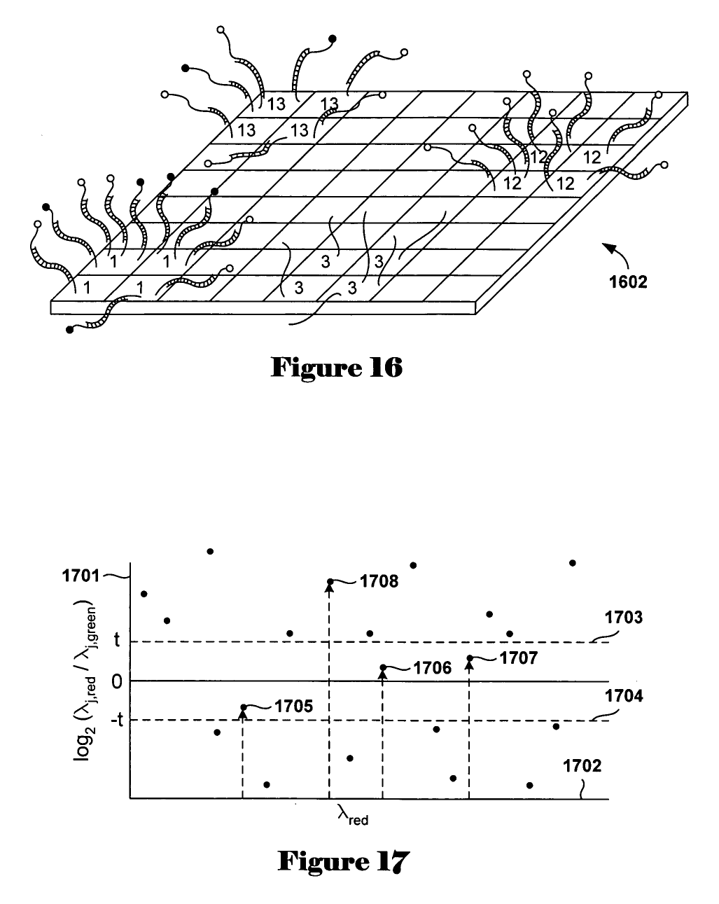

When microarrays are exposed to a sample solution, target molecules are allowed to hybridize through nucleotide pairing interactions with complementary sequences of candidate probes bound to the surface of the microarray. FIG. 16 illustrates a hypothetical replicate microarray after exposure to hypothetical sample solution 1505, described above in FIG. 15. In FIG. 16, four groups of four replicate features of the microarray 1502 are singled out in order to illustrate four of many possible outcomes of a microarray-based hybridization assay. Replicate features labeled “1” show the idealized result for a candidate probe that may be functional as a dye-normalization probe, because an even distribution of red-labeled and green-labeled bound target molecules suggests that the candidate probe molecules have a nucleotide sequence [NS]ₙ that is complementary to nucleotide sequences of both lung and heart target molecules. The replicate features labeled “13” show an uneven distribution of bound target molecules. This probe sequence may or may not be functional for the target-molecule pair depending on the tolerance described below. The candidate probe molecules bound to replicate features labeled “12” hybridize only with heart target molecules, suggesting that the lung target does not contain nucleotide molecules complementary to the candidate probe molecules at feature label “12.” Lastly, the empty replicate features labeled “3” suggest that these candidate probes do not contain a nucleotide sequence complementary to either lung or heart target molecules.

The replicate microarrays are then read and the image data analyzed to determine those candidate probes that are functional across tissue and species. One of many possible means for analyzing the functionality of candidate probes is to plot the intensity log ratio versus red and green signal intensity. The log ratio for each target-molecule pair experiment is computed according to the following expression:

$$Equation ( 5 ) \text{:}$$

$${log}_{2} ( \frac{{λ}_{j , red}}{{λ}_{j , green}} )$$

where j=the replicate feature index;
λ_{j,red}=the red intensity wavelength of replicate features j; and
λ_{j,green}=the green intensity wavelength of replicate features j.
FIG. 17 illustrates a log ratio data plot of a hypothetical target-molecule pair. In the plot, the vertical axis 1701 corresponds to the log ratio given by equation (5), and the horizontal axis 1702 corresponds to λ_{j,red}. A tolerance interval is used to determine which candidate probe molecules are considered capable of hybridization with both target molecules of a target-molecule pair. The tolerance interval is determined by:

$$Equation ( 6 ) \text{:} \\ - t < {log}_{2} ( \frac{{λ}_{j , red}}{{λ}_{j , green}} ) < t$$
where t=tolerance.
   In FIG. 17, dashed lines 1703 and 1704 identify a hypothetical tolerance interval according to equation (6). Data points that fall within the tolerance interval, such as data points 1705-1707, exhibit nearly equal red and green intensity wavelengths and identify candidate probe molecules that have nucleotide sequences complementary to labeled target-molecule pairs. On the other hand, data points, such as data point 1708, that are outside the tolerance interval identify candidate probe molecules that show a preference for hybridization with target molecules of one tissue over the other. The candidate probe molecules the exhibit log ratios within a tolerance interval for all target molecule pairs of a particular species are said to “functional across tissues,” and candidate probe molecules that are function across tissues for all selected species are said to be “functional across species.” For example, the candidate probes that exhibit log ratios within the tolerance interval in all 10 target-molecule pairs listed in Table 2, are “functional across tissues” of any species listed in Table 1. The candidate probes that are functional across tissues for all 10 species listed in Table 1, are “functional across species.”

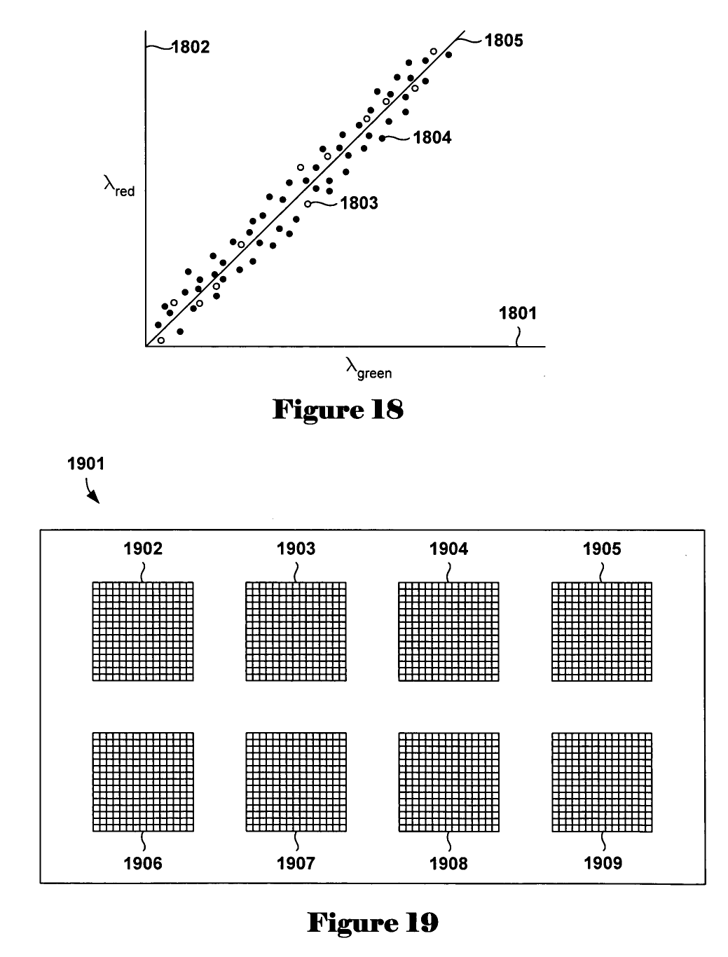

Next, the candidate probe molecules that satisfy the tolerance interval requirements described above with reference to FIG. 17 and span as much as possible the intensity distribution range of any microarray experiment compose the set of dye-normalization probes. In other words, the entire intensity distribution may be divided into three segments (low, medium, and high), and the number of candidate probe molecules in each segment is comparable. FIG. 18 illustrates a red-signal intensity to green signal intensity plot for a hypothetical set of candidate probe molecules that span the intensity range of any microarray-based hybridization assays and satisfy the tolerance requirements of equation (6). In FIG. 18, the horizontal axis 1801 corresponds to green signal intensities, and the vertical axis 1802 corresponds to red signal intensities. Because the data points, such as data points 1803 and 1804, have log ratios close to zero, the data points are in close proximity to the central tendency line 805 having slope 1. The set of open data points, such as data point 1803, correspond to dye-normalization probes that can be selected to normalize the intensities distribution of other microarray-based hybridization assays, because these data points represent probes that span the intensity range of any microarray-based hybridization assays and satisfy the tolerance criteria described above with reference to FIG. 17.

In order to determine candidate probe molecules that are suitable for a variety of hybridization conditions, the sample solution conditions, such as temperature, acidity, alkalinity, and salinity, may be varied for hybridization assays having one or more identical sample solutions. Each condition can varied without variation of the other conditions. For example, in order to determine which candidate probe molecules are functional for a variety of hybridization temperatures, candidate probe molecules are tested by hybridizing identical sample solutions at different hybridization temperatures, such as 50°, 55°, 60°, 65° and 70° Celsius. Moreover, combinations of the conditions can be varied, such as varying the temperature and acidity.

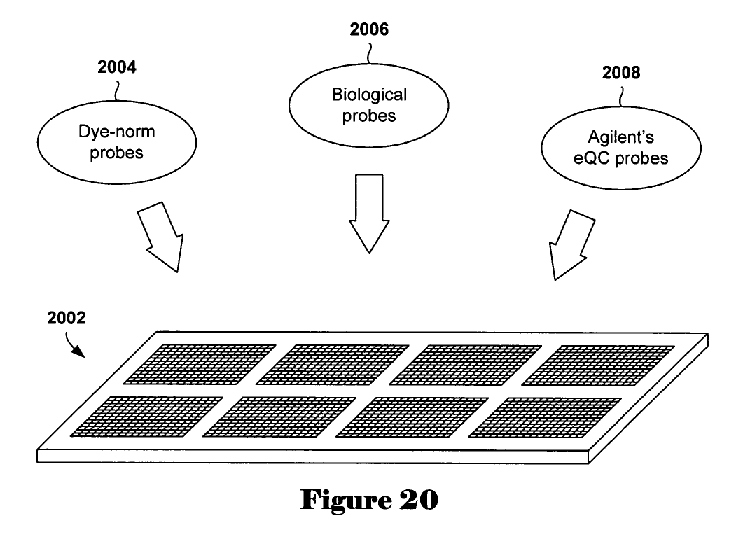

The surviving set of candidate probe molecules that satisfy the tolerance requirements, as described above with reference to FIG. 17, and span the entire intensity range, as describe above with reference to FIG. 18, compose the set of dye-normalization probes. However, the functionality of the probes composing the set of dye-normalization probes may be experimentally validated using multiple arrays on a common substrate, such as low-feature-number microarrays or 8-pack microarrays. FIG. 19 is an illustration of an example 8-pack of microarray 1801 having eight microarrays 1902-1909. Three separate 8-pack microarray feature arrangements are designed, one for each of the species “Human,” “Mouse,” and “Rat.” FIG. 20 illustrates three kinds of probes employed in each of the three probe designs. In FIG. 20, the hypothetical 8-pack microarray 2002 is arrayed with approximately 300 probes selected from the set of dye-normalization probes 2004, as described above with reference to FIGS. 9-18, a set of randomly-selected, high-quality biological probes for each species 2006, and a set of Agilent's embedded quality control (“eQC”) probes 2008. Note that for the “Human,” “Mouse,” and “Rat” 8-pack microarray designs, the randomly-selected, high-quality biological probes are intended for hybridization with “Human,” “Mouse,” and “Rat” target molecules, respectively.

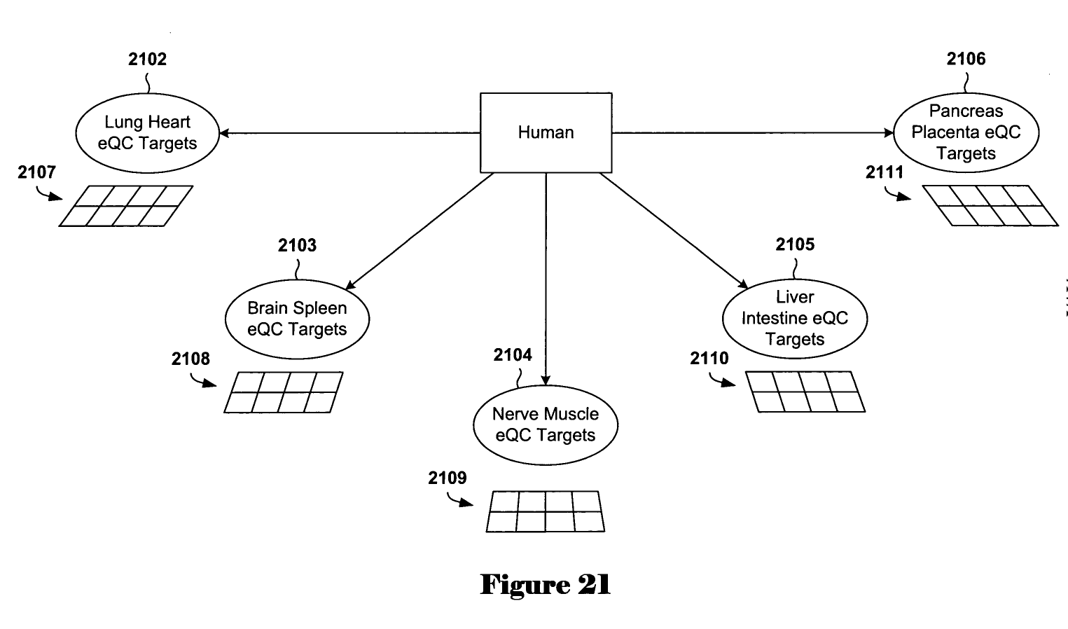

Five separate sample solutions composed of tissues pairs isolated from each of the three species “Human,” “Mouse,” and “Rat” are prepared. For example, the hypothetical target-molecule pairs, described above with reference to Table 2, can be isolated for each species. The five different samples solutions composed of target-molecule pairs are spiked with synthetic targets that are complementary to the eQC probes, for which different expression results have already been determined. The synthetic target molecules are referred to as “eQC target molecules.” FIG. 21 illustrates five hypothetical sample solutions prepared for validating approximately 300 probes selected from the set of dye-normalization probes using identical 8-pack microarrays designed for the “Human” species 2101. In FIG. 21, hypothetical sample solutions 2102-2106 are composed of target-molecule pairs and eQC target molecules. For example, sample solution 2102 is composed of lung, heart and eQC target molecules. The lung and heart target molecules are labeled as describe above with reference to FIG. 15, and the eQC target molecules are labeled with a predetermined expression result in mind. The five separate hypothetical microarray-based hybridization assays shown in FIG. 21 are performed by exposing each of the identically designed 8-pack microarrays 2107-2111 to one of the sample solutions 2102-2106, respectively. The method described with reference to FIGS. 20 and 21 is repeated for the “Mouse” and “Rat” species.

After the 8-pack microarray-based hybridization assays are completed, the data is examined using Agilent's Feature Extraction software described in detail in U.S. Pat. No. 6,591,196, entitled “Method and System for Extracting Data from Surface Array Deposited Features,” filed Jun. 6, 2000, which is incorporated by reference. For each 8-pack microarray, approximately 300 dye-normalization probes are used to normalize the intensity data using the “Norm file editor” method in Agilent's Feature Extraction software. The log ratio results to be derived from each eQC probe are known. The effectiveness of the dye-normalization probes in normalizing 8-pack microarray data is indicated by the accuracy of the differential expression values generated from the eQC probes. The data normalized using the 300 dye-normalization probes is compared to data from identical microarrays that have been normalized using Agilent's standard rank consistency dye-normalization method described in U.S. Patent Application No.: U.S. 2003/0215807, entitled “Method and System for Normalization of Microarray Data Based on Local Normalization of Rank-Ordered, Globally Normalized Data,” filed May 9, 2002, which is incorporated by reference.

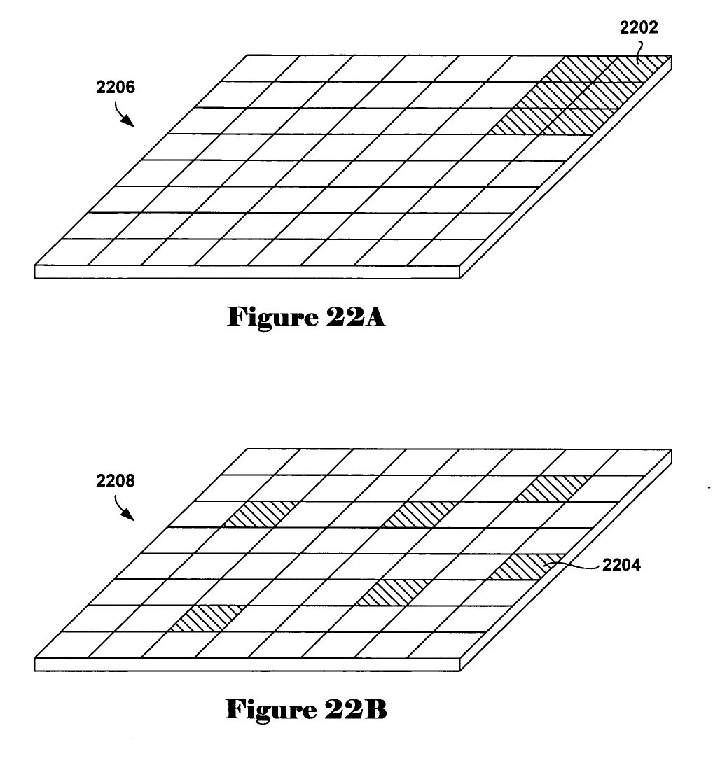

A subset of the set of dye-normalization probes can be used to normalize the signal data from a variety of microarray experiments by dedicating approximately 10% of the features of a microarray to dye-normalization probes. FIGS. 22A-B show two of many possible dye-normalization probe feature arrangements for two hypothetical microarrays. In FIGS. 22A-B, the dye normalization probes are identified by shaded square features, such as features 2202 and 2204, respectively. In FIG. 22A, 6 adjacent features in the top, right-hand corner of the hypothetical microarray 2206 are dedicated to synthetic dye-normalization probes. In FIG. 22B, 6 features of hypothetical microarray 1708 dedicated to synthetic dye-normalization probes have been randomly selected.

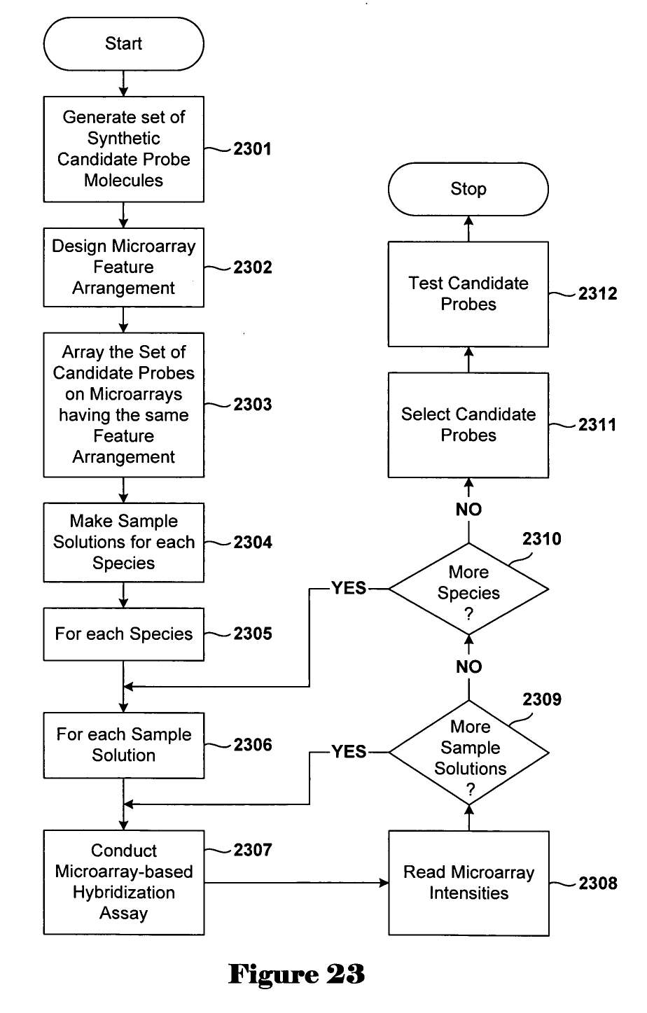

FIG. 23 is a control-flow diagram that represents one of many possible methods according to the present invention for determining a set of synthetic dye-normalization probes. In step 2301, a set of candidate probes is generated, as described above in relation to FIG. 10. In step 2302, the microarray feature arrangement of the candidate probe molecules is designed. In step 2303, candidate probe molecules are arrayed on a set of microarrays having identical, synthetic candidate probe molecule feature arrangements, as described above with reference to FIG. 11. In step 2304, sample solutions for each species are prepared, as described above in relation to Tables 1 and 2 and FIGS. 13 and 14. In outer for-loop of step 2305, steps 2306-2310 are repeated for each species. In inner for-loop of step 2306, steps 2307-2309 are repeated for each sample solution. In step 2307, microarray-based hybridization assays are conducted, as described above in relation to FIGS. 14 and 15. In step 2308, the signal intensities for each microarray are read and stored. In step 2309, if the set of sample solution is not exhausted, then step 2307 and 2308 are repeated. Otherwise, control proceeds to step 2310. In step 2310, if the set of chosen species is not exhausted, then steps 2306-2309 are repeated. Otherwise, control proceeds to step 2311. In step 2311, candidate probe molecules are selected, as described above with reference to FIGS. 17 and 18. In step 2312, the set of candidate probe molecules selected in step 2311 are tested, as described above with reference to FIGS. 19-21.

Although the present invention has been described in terms of a particular embodiment, it is not intended that the invention be limited to this embodiment. Modifications within the spirit of the invention will be apparent to those skilled in the art. For example, an almost limitless number of different implementations of the many possible embodiments of the method of the present invention can be performed. In alternate embodiments, features in alternative types of molecular arrays may be arranged to cover the surface of the molecular array at higher densities, such as offsetting the features in adjacent rows in order to produce a more densely packed feature arrangement. In alternate embodiments, one, three, four or more tissues can be used in an experiment to determine functional candidate probes that span tissues of a single species. In alternate embodiments, the number of tissues pairs selected from a single species can range from about 2 to 16 or 20 or more different tissues, and can include diseased tissues, such as leukemia, HeLa, MG63, and K-562 cells. In an alternate embodiment, the steps used to determine the set of dye-normalization probes described above with reference to FIGS. 9-18 can be repeated using dye-swap microarray-based hybridization assays to validate the set of candidate probe molecules. In alternate embodiments, the candidate probe molecules that consistently have a log ratio close to “0,” and consistently span the entire intensity distribution of any given microarray experiment across all target-molecule pairs for a particular species, such as “Human,” “Mouse,” or “Rat,” compose the set of synthetic dye-normalization probes.

The foregoing description, for purposes of explanation, used specific nomenclature to provide a thorough understanding of the invention. However, it will be apparent to one skilled in the art that the specific details are not required in order to practice the invention. The foregoing description of specific embodiments of the present invention are presented for purposes of illustration and description. They are not intended to be exhaustive or to limit the invention to the precise forms disclosed. Obviously many modifications and variations are possible in view of the above teachings. The embodiments are shown and described in order to best explain the of the invention and its practical applications, to thereby enable others skilled in the art to best utilize the invention and various embodiments with various modifications as are suited to the particular use contemplated. It is intended that the scope of the invention be defined by the following claims and their equivalents:

What is claimed is:

1. A method for determining a set of dye-normalization probes, the method comprising:
generating a set of candidate probe molecules;
arraying the set of candidate probe molecules on one or more replicate microarrays;
making sample solutions from one or more tissues of one or more species;
conducting microarray-base hybridization assays using the one or more replicate microarrays for each sample solution; and
determining a subset of the candidate probe molecules that are functional for the microarray-based hybridization assays.

2. The method of claim 1 wherein generating the set of candidate probe molecules further includes:
synthesizing candidate probe molecules having low-complexity, low-entropy nucleotide sequences;
employing candidate probe molecules having low-complexity, low-entropy nucleotide sequences that are complementary to sequenced target molecule; and
employing candidate probe molecules having lengths ranging from about 25 to about 60 or more nucleotides.

3. The method of claim 1 wherein the replicate microarrays further includes microarrays having similar feature arrangements.

4. The method of claim 3 wherein the replicate microarrays further include one or more features having similar bound candidate probe molecules.

5. The method of claim 1 wherein making the sample solutions from one or more tissues of one or more species further includes:
isolating a set of target molecules for each tissue;
labeling members of each set of target molecules with identical signal emitting labels; and
labeling each set of target molecules with different signal emitting labels.

6. The method of claim 5 wherein isolating the set of target molecules for each tissue further includes amplifying expressed messenger RNA molecules for each tissue.

7. The method of claim 5 wherein isolating the set of target molecules for each tissue further includes amplifying complementary DNA molecules for each tissue.

8. The method of claim 1 wherein conducting the microarray-base hybridization assays further includes varying one or more of:
temperature;
acidity;
alkalinity; and
salinity.

9. The method of claim 1 wherein determining the subset of candidate probe molecules that are functional a wide range of microarray-based hybridization assays further comprising:
selecting candidate probe molecules that hybridize with target-molecule pairs extracted from different species, tissues, and under varying hybridization conditions;
selecting candidate probe molecules having log ratios within a tolerance interval about zero; and
selecting candidate probe molecules having signal intensities that span the entire intensity distribution range of the microarray-based hybridization assays.

10. The method of claim 1 wherein determining the set of candidate probe molecules further includes validating the subset of candidate probe molecules using multiple arrays on a common substrate.

11. The method of claim 10 wherein validating the subset of candidate probe molecules further includes arraying approximately 300 of the subset of candidate probe molecules, a quantity of randomly-selected, high-quality biological probes, and a quantity of quality control probes on the multiple arrays on a common substrate.

12. The method of claim 11 wherein the randomly-selected, high-quality biological probes further includes probes isolated from tissues of about two or more species.

13. Transferring results produced by a microarray reader or microarray data processing program employing the method of claim 1 stored in a computer-readable medium to an intercommunicating entity.

14. Transferring results produced by a microarray reader or microarray data processing program employing the method of claim 1 to an intercommunicating entity via electronic signals.

15. A method comprising forwarding data produced by employing the method of claim 1 to a remote location.

16. A method comprising receiving data produced by employing the method of claim 1 from a remote location.

17. A system for determining a set of microarray probes, the system comprising:
a computer processor;
a communications medium by which microarray data are received by the microarray-data processing system;
a program, stored in the one or more memory components and executed by the computer processor that generates nucleotides sequences for a set of candidate probe molecules; arrays the set of candidate probe molecules on one or more replicate microarrays; and determines a subset of the candidate probe molecules that are functional for two or more tissues of two or more species.

18. The system of claim 17 wherein determines the subset of candidate probe molecules further includes determines a log ratio of the one or more sample solutions.
